(19) United States
(12) Patent Application Publication
(10) Pub. No.: US 2022/0192937 A1
(43) Pub. Date: Jun. 23, 2022

(54) PORTION UNIT WITH COSMETIC PRODUCT FOR OXIDATIVE COLOR CHANGE OF KERATINOUS FIBERS

(71) Applicant: Henkel AG & Co. KGaA, Duesseldorf (DE)

(72) Inventors: Udo ERKENS, Willich (DE); Torsten LECHNER, Langenfeld (DE)

(73) Assignee: Henkel AG & Co. KGaA, Duesseldorf (DE)

(21) Appl. No.: 17/553,146

(22) Filed: Dec. 16, 2021

(30) Foreign Application Priority Data
Dec. 18, 2020 (DE) 102020134100.8

(51) Int. Cl. A61K 8/19; A61K 8/02; A61K 8/81; A61K 8/25; A61K 8/41; A61K 8/73; A61Q 5/08; A61Q 5/10

(52) U.S. Cl. CPC A61K 8/19; A61K 8/0208; A61K 8/8129; A61K 8/25; A61K 8/411; A61K 2800/432; A61K 8/731; A61K 8/73; A61Q 5/08; A61Q 5/10; A61K 2800/48; A61K 8/415

(57) ABSTRACT

The present disclosure relates to a portion unit with at least one cosmetic product for oxidative color change of keratinous fibers, in particular human hair, filled receiving chamber, which is formed by a water-soluble envelope made of a water-soluble nonwoven fabric. Another object of the present disclosure is a method for oxidative dyeing of keratinous fibers, in particular human hair, using the portion unit.

### CROSS-REFERENCE TO RELATED APPLICATION

This application claims priority to German Patent Application No. 102020134100.8, filed Dec. 18, 2020, which is incorporated herein by reference in its entirety.

### TECHNICAL FIELD

The present disclosure relates to a portion unit with at least one cosmetic product for oxidative color change of keratinous fibers, in particular human hair, filled receiving chamber, which is formed by a water-soluble envelope made of a water-soluble nonwoven fabric. Another object of the present disclosure is a method for oxidative dyeing of keratinous fibers, in particular human hair, using the portion unit.

### BACKGROUND

Changing the color of keratin fibers, especially hair, is a key area of modern cosmetics. As a result, the appearance of the hair can be adapted both to current fashion trends and to the individual wishes of the individual. The expert knows different possibilities for changing the hair color.

The hair color can be changed temporarily by using direct dye. Here, already fully formed dyes from the dye diffuse into the hair fiber. The dyeing with direct dyes is associated with slight damage to the hair, but a disadvantage is the short shelf life and the quick washability of the dyeing is obtained with direct dyes.

Therefore, if the consumer desires a long-lasting color result or a shade lighter than his or her original hair color, oxidative color modifiers are commonly used. For permanent, intensive dyeing with corresponding fastness properties, so-called oxidation dyes are used. Such colorants usually include oxidation dye precursors, so-called developer components and coupler components, which form the actual dyes with one another under the influence of oxidizing agents—usually hydrogen peroxide. Oxidation dyes are exemplified by excellent, long-lasting dyeing results.

Pure lightening or bleaching of hair is often done by using oxidizing agents without the addition of oxidation dye precursors. For a medium bleaching effect, the use of hydrogen peroxide alone as an oxidizing agent is sufficient; to achieve a stronger bleaching effect, a mixture of hydrogen peroxide and peroxide sulfate salts is usually used.

Oxidative color change agents usually come on the market in the form of two-component agents, in which two different preparations are packaged separately in two separate packages and are only mixed with one another shortly before use.

The first preparation is a formulation—usually acidic for stability reasons—which comprises, for example, liquid hydrogen peroxide in concentrations of 1.5 to 12% by weight as oxidizing agent. The oxidizing agent formulation is usually in the form of an emulsion or dispersion and is usually provided in a plastic bottle with a resealable outlet opening (developer bottle).

This oxidizing agent formulation is mixed with a second preparation prior to use. This second preparation is an alkaline formulation which is often in the form of a cream or a gel and which, if a color change is also desired at the same time as the lightening, also comprises at least one oxidation dye precursor. This second preparation may be provided, for example, in the form of a tube or in the form of a plastic or glass container.

In the usual form of application described above, the second preparation comprising the alkalizing agent and/or the oxidation dye precursors is transferred from the tube or container into the developer bottle and then mixed by shaking with the hydrogen peroxide preparation already in the developer bottle. In this way, the application mixture is produced in the developer bottle. Application to the hair is then done via a small spout or outlet on the head of the developer bottle. The spout or outlet is opened after shaking and the application mixture can be removed by pressing the flexible developer bottle.

When preparing the application mixture in a bowl, both components—the first preparation comprising the oxidizing agent and the second preparation comprising alkalizing agent and/or oxidation dye precursors—are completely transferred to a bowl or similar vessel and mixed there, for example with the aid of a brush. The application mixture is then removed from the mixing tray using the brush. With this form of application, the use of a voluminous and expensive developer bottle is not necessary.

However, the filling of oxidizing agent preparations into such packaging is associated with problems, the cause of which lies in the reactivity of the oxidizing agent. Oxidizing agents are highly reactive substances, usually in liquid or paste form, which—depending on the storage conditions and on the presence of decomposing impurities—decompose in small proportions to form oxygen (i.e., gas).

The developer bottles known from the prior art are only filled to a maximum of half, usually only a third of their inner volume with the oxidizing agent composition. As a rule, developer bottles are made of polyethylene. Since polyethylene is permeable to both water vapor and gases, there is no or little excess pressure in the developer bottle. In addition, developer bottles are usually provided with sturdy, thick walls and a stable screw cap, so that diffusion of the water vapor or gases is reduced by the thickness of the walls and a pressure increase within the bottle that occurs to a small extent has no negative effects.

As a result, the packaging is usually bulky, which compromises sustainability in terms of environmental protection and resource conservation.

### BRIEF SUMMARY

This disclosure provides a portion unit comprising at least one receiving chamber which is formed by a water-soluble envelope, wherein the water-soluble envelope comprises a water-soluble nonwoven fabric and wherein the receiving chamber comprises a cosmetic product for the oxidative color change of keratinous fibers, the cosmetic product comprising (i) at least one oxidation composition (OC) comprising at least one solid oxidizing agent as oxidizing compound, and (ii) at least one cosmetic coloring composition (DC).

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

By using solid oxidation compositions, handling can be simplified at least to the extent that no mixing with hydrogen peroxide is necessary, but the solid oxidation composition can be mixed with water and thus activated. However, when dosing the oxidation composition, make sure that the amount used matches the dyeing composition to achieve a correct dyeing result. There is therefore a need for easy handling of dyeing compositions, i.e., cosmetic products for oxidative color change of keratinous fibers, especially human hair.

Surprisingly, it was found that this task is solved by providing a cosmetic product for oxidative color change of keratinous fibers, especially human hair, in a portion unit formed of a water-soluble nonwoven fabric.

Therefore, a first object of the present disclosure is a portion unit comprising at least one receiving chamber formed by a water-soluble envelope, wherein the water-soluble envelope comprises or consists of a water-soluble nonwoven fabric and wherein the receiving chamber comprises a cosmetic product for oxidative color change of keratinous fibers, in particular human hair.

A further object of the present disclosure is a process for the oxidative dyeing of keratinous fibers, in particular human hair, using a portion unit as contemplated herein. Keratinic fibers, keratin comprising fibers or keratin fibers are to be understood as furs, wool, feathers and in particular human hair. Although the agents as contemplated herein are primarily suitable for lightening and dyeing keratin fibers, in principle there is nothing to prevent their use in other areas.

Prefabrication enables a defined application quantity to be made available when using the cosmetic product for oxidative color change of keratinous fibers, in particular human hair, so that the risk of misapplication due to incorrect mixing ratios is minimized. The portion unit as contemplated herein is mixed with water before application and can be applied after reaching a sufficient application viscosity. In storage tests, the nonwoven proved to be more stable in storage than commercially available water-soluble films. In the case of water-soluble PVA films, storage tests led to embrittlement of the film, which was reflected in a reduced solubility behavior of the film. In addition, it also leads to increased fragility of the sachet. These disadvantages are also solved by the portioning unit as contemplated herein. By providing the cosmetic product in a water-soluble wrapper as contemplated herein, simple and uncomplicated handling of the product is made possible while avoiding additional packaging waste. This allows the agent to be easily converted to a ready-to-use state without the need for additional portioning. In addition, by using its nonwoven material as the wrapping/portioning unit, the end customer has a tactile soft feeling when using it.

### Water Soluble Coating

The portion unit as contemplated herein comprises or consists of at least one receiving chamber formed by a water-soluble envelope comprising or consisting of a water-soluble nonwoven fabric.

The term nonwoven is used to describe structures made of fibers that are joined together to form a fiber layer. The nonwovens are preferably flexible and pliable. Films or papers are not counted as nonwovens.

In a preferred embodiment of the portion units, the water-soluble nonwovens are needled nonwovens. In corresponding nonwovens, entanglement and thus compaction and consolidation of the fibers is achieved by needling, for example by employing suitable needle boards or needle bars.

Preferred nonwovens, preferably needle-punched nonwovens, are those from the group of random nonwovens. In these nonwovens, the fibers have any direction and are evenly distributed in all directions of the nonwoven. Corresponding nonwovens are not only exemplified by a special look and feel, but also improve the mechanical stability of the cosmetic product encompassed by the portion unit during transport and storage due to their compressibility.

Regarding the optical, haptic and mechanical properties of the portion unit, it has proved advantageous if the water-soluble nonwoven, in particular the random nonwoven, has a basis weight of from about 20 to about 200 g/m², preferably from about 30 to about 100 g/m² and from about 35 to about 60 g/m². A higher basis weight results in a higher stability of the coating but reduces the solubility in water or prolongs the duration of the dissolution of the coating.

Due to their properties, water-soluble nonwovens from the group of cross-layer nonwovens are particularly preferred in addition to the random-layer nonwovens. These nonwovens are produced, for example, by laying down the fibers on a laying-down belt with a longitudinal orientation of the fibers to the overall nonwoven, the fibers preferably being oriented in two directions.

Particularly preferably, the water-soluble nonwoven fabric is a non-woven, cross-laid and spot-sealed nonwoven fabric, which is preferably single-ply.

When using such cross laid nonwovens, it has proved advantageous if the water-soluble nonwoven, the cross laid nonwoven, has a basis weight of about 20 to about 80 g/m², preferably about 35 to about 70 g/m², particularly preferably about 38 to about 60 g/m².

In a further preferred embodiment, the water-soluble nonwoven fabric is a woven nonwoven fabric. When using a woven water-soluble nonwoven, very satisfactory results were obtained, particularly regarding the mechanical strength and tightness of the portion unit. In embodiments in which a woven nonwoven fabric is used, the nonwoven fabric preferably has a basis weight of about 35 to about 160 g/m², preferably about 80 to about 120 g/m² or about 35 to about 80 g/m² or about 120 to about 160 g/m². In another preferred embodiment of the portion unit, the water-soluble nonwovens are cloth-knit nonwovens. In these nonwovens, the bonding of the fibers is achieved by fabric closure using additives. These binders enable the fibers to be adhesively bonded to each other.

Preferred nonwovens are based on water-soluble polymer fibers, in particular water-soluble fibers based on polyvinyl alcohol and polyvinyl alcohol copolymers. In a preferred embodiment, the water-soluble nonwoven comprises at least about 50% by weight, preferably at least about 70% by weight and in particular at least about 98% by weight of polyvinyl alcohol or polyvinyl alcohol copolymers, in particular polyvinyl alcohol, the weight percentages in each case being based on the total weight of the water-soluble coating. In addition to good water solubility, fibers based on polyvinyl alcohols and polyvinyl alcohol copolymers have the advantage that they are biodegradable and can thus be returned to the value-adding cycle. The degree of saponification of the polyvinyl alcohol is preferably about 80 to about 100%, preferably about 84 to about 99%, more preferably about 87 to about 98%, more preferably about 90 to about 97%, more preferably about 91 to about 96%, more preferably about 92 to about 95%, more preferably about 93 to about 94%.

The fiber length of the water-soluble polymer fibers is preferably in the range from about 1 to about 40 mm, particularly preferably from about 2 to about 20 mm. The thickness of the fibers is preferably about 0.1 to about 1000 μm, preferably about 0.5 to about 400 μm. The density of the nonwoven is preferably above about 0.15 g/cm³, preferably above about 0.2 g/cm³ and in the range of about 0.2 to about 0.8 g/cm³.

The nonwovens can be produced, for example, by employing mechanical, aerodynamic or hydrodynamic processes of the prior art. The fibers of the nonwoven can be bonded mechanically, for example by frictional or positive locking, chemically, for example using binders or solvents, or thermally, using thermoplastic additives.

To improve the stability and tightness of the portion unit, the water-soluble wrapping of the portion unit as contemplated herein preferably further comprises a water-soluble polymer film, the film preferably being a film of polyvinyl alcohol. In a particularly preferred embodiment, the water-soluble nonwoven fabric is coated with the water-soluble film, in particular the side of the nonwoven fabric that meets the cosmetic agent during intended use. In this way, the tightness of the portion unit as contemplated herein can be further improved so that the portion unit can also be used and stored without problems in connection with liquid or gel cosmetic products.

There are no specific requirements for the shape and size of the portion unit. Rather, the shape and size can be adjusted as needed. In a preferred embodiment, the portion unit is formed entirely by the receiving chamber. In an alternative preferred embodiment, the portion unit has several, preferably separate, receiving chambers.

The surface of the portion unit can be used to make the portion unit visually appealing. Thus, in a preferred embodiment, the portion unit has a printed surface.

### Cosmetic Product and Method

As contemplated herein, the water-soluble coating comprises, in at least one receiving chamber, a cosmetic product for oxidative color change of keratinous fibers, in particular human hair. The cosmetic product comprises (i) at least one oxidation composition (OC), anhydrous, comprising at least one solid oxidizing agent as oxidizing compound, and (ii) at least one cosmetic dyeing composition (DC).

The cosmetic product is in the form of a powder (such as a coloring powder). In the context of the present disclosure, the terms “powder” or “powdery” are understood to mean those agents which include comminuted solid constituents, wherein the comminution may be achieved by trituration, crushing, grinding or by atomization-drying or freeze-drying. Thus, a powder is a mixture comprising small, solid particles. Powders can be composed of solid components with different particle sizes. Typically, however, it may be preferred if the powders have a particle size that is as homogeneous as possible, particularly to facilitate uniform dispersion or dissolution of the powders in water. A preferred powder in the sense of the present disclosure has an average particle diameter of at least about 20 μm and a BET surface area of about 40 to about 400 m²/g.

Another object of the present disclosure is a method for oxidative dyeing of keratinous fibers, in particular human hair, comprising the following steps:

(i) Providing a wrapper as contemplated herein with a cosmetic product as previously described,

(ii) Mixing of the coating with water to a ready-to-use colorant,

(iii) directly following step (ii), applying the ready-to-use colorant to keratinous fibers, to human hair,

(iv) Leaving the ready-to-use colorant on the keratinous fibers, on human hair, for about 5 to about 60 minutes, preferably for about 15 to about 45 minutes, particularly preferably for about 30 minutes,

(v) then rinsing the colorant from the keratinous fibers with water, optionally washing the fibers with a surfactant-containing detergent, conditioning the fibers with a conditioning agent, and/or drying the fibers, wherein the weight ratio of the cosmetic product to the weight of the amount of water used in step (ii), is in the range of about 16:100 to about 25:100, preferably about 16:100 to about 24:100, particularly preferably about 16:100 to about 22:100, more preferably about 16:100 to about 20:100, further particularly preferably about 16:100 to about 19:100, exceptionally preferably about 16:100 to about 18:100.

As contemplated herein, oxidation composition (OC) and dyeing composition (DC) may be present in a receiving chamber of the envelope. However, as contemplated herein, it is also possible for an envelope to have two or more receiving chambers and to have the oxidation composition (OC) in one receiving chamber and the dyeing composition (DC) in a second receiving chamber separate therefrom. As contemplated herein, it is also possible that an envelope with one receiving chamber comprises the oxidation composition (OC) and a second receiving chamber different therefrom comprises the dyeing composition (DC), and these two envelopes are in the form of a kit.

The physical state “solid”, as far as described in the present application, refers to standard conditions, i.e., about 20° C. and about 10⁵ Pa. Unless otherwise stated, all data concerning the state of aggregation refer to these standard conditions.

The cosmetic product for oxidative color change of keratinous fibers, in particular human hair, included in the portion unit as contemplated herein is preferably anhydrous, i.e., it has a water content of about 0 to about 8% by weight, preferably about 0.1 to about 5% by weight, particularly preferably about 0.5 to about 2.5% by weight, based on its weight, as contemplated herein. With the above-mentioned water contents in the range from about 0 to about 8% by weight, the cosmetic product portioned as contemplated herein is anhydrous within the meaning of the present application. These data refer to the free water content. The content of molecularly bound water or water of crystallization, which individual powder components may have, is not considered. The water content can be measured with a moisture analyzer or moisture meter, for example a moisture meter from Mettler, model Mettler HS 153, where the loss on drying is determined at about 105° C., shut-off criterion about 50 seconds with a product weight of about 1 to about 1.5 grams.

The cosmetic product in the envelope as contemplated herein is a product for the oxidative coloration of keratinous fibers, which is to say, a product applied on the human head to obtain an oxidative coloration or a nuance of the hair. In this context, shading is understood to mean coloring in which the color result is lighter than the original hair color. Throughout this application, the term “cosmetic dyeing composition (DC)” is used synonymously with the terms “preparation (DC)” or “coloring composition (DC).”

Preferred embodiments of the oxidation composition (OC) and the coloring composition (DC) are further detailed below.

The product as contemplated herein comprises an oxidation composition (OC), anhydrous, comprising at least one solid oxidizing agent. The oxidizing agent is selected from percarbonates, perborates, percarbamides or mixtures thereof. Particularly preferably, the oxidizing agent is selected from percarbonates, perborates or mixtures thereof.

A percarbamide is an addition compound of hydrogen peroxide and urea. By a percarbonate shall preferably be understood an H₂O₂ adduct. Furthermore, a perborate, in particular sodium perborate is used as a solid oxidizing agent.

In a particularly preferred embodiment, the solid oxidizing agent is sodium percarbonate. Sodium percarbonate is understood to be an H₂O₂ adduct with the formula 2 Na2CO₃.3H₂O₂. When mixed with the dyeing composition (DC), which may comprise an alkalizing agent, and water, the ready-to-use dyeing agent is formed, in which the sodium percarbonate develops its effect as an oxidizing agent.

In a further preferred embodiment, the solid oxidizing agent is at least one perborate. Perborates are borates in which one oxygen atom is replaced by a dioxygen group. Perborates as contemplated herein are inorganic compounds. A particularly suitable perborate is sodium perborate.

Sodium perborate is alternatively called sodium peroxoborate. The sodium perborate as contemplated herein is the commercially available sodium peroxoborate tetrahydrate (sodium perborate tetrahydrate) with the empirical formula (NaBO₃.4H₂O). Alternatively, the sum formula NaBO₂ can be found in the literature.H₂O₂.3H₂O. In the solid state, ring-shaped peroxoborates are present, with the formula Na₂B₂(O₂)₂(OH)₄.6H₂O. Sodium perborate tetrahydrate has the CAS No. 10486-00-7 and is commercially distributed, for example, by the company Sigma Aldrich.

To achieve optimum color results, the solid oxidizing agent(s), in percarbonates or perborates, are preferably used in certain ranges of amounts in the cosmetic product as contemplated herein. Particularly satisfactory results were obtained when the product comprises—based on its total weight—about 0.5 to about 14.0% by weight, preferably about 1.0 to about 12.0% by weight, more preferably about 1.2 to about 10.0% by weight, still more preferably about 1.4 to about 8.0% by weight and very preferably about 2.0 to about 5.0% by weight of solid oxidizing agent, in percarbonate(s) or perborate(s).

In a particularly preferred embodiment, a cosmetic product as contemplated herein is wherein it comprises—based on its total weight—about 0.5 to about 14.0% by weight, preferably about 1.0 to about 12.0% by weight, further preferably about 1.2 to about 10.0% by weight, still further preferably about 1.4 to about 8.0% by weight and very particularly preferably about 2.0 to about 5.0% by weight of sodium perborate or sodium percarbonate.

In a preferred embodiment, the at least one anhydrous cosmetic dyeing composition (DC) comprises at least one developer-type oxidation dye precursor (developer component) and at least one coupler-type oxidation dye precursor (coupler component). Developer components and coupler components are used in molar amounts to each other. Although molar use has been shown to be appropriate, a certain excess of individual oxidation dye precursors is not detrimental, so that developer components and coupler components can be used in a molar ratio of about 3:1 to about 1:3, especially about 2:1 to about 1:1, may be included.

Developer components preferred as contemplated herein are selected from the group formed by toluene-2,5-diamine, 2-methoxymethyl-p-phenylenediamine, 2-(2,5-diaminophenyl)ethanol, p-phenylenediamine, 2-(1,2-dihydroxyethyl)-p-phenylenediamine, N,N-bis-(2-hydroxyethyl)-p-phenylenediamine, 4,5-diamino-1-(2-hydroxyethyl)-1H-pyrazole, 4-aminophenol, 3-methyl-4-aminophenol, N-(4-amino-3-methylphenyl)-N-[3-(1H-imidazol-1-yl)propyl]amine, N,N′-bis-(2-hydroxyethyl)-N, N′-bis-(4-aminophenyl)-1,3-diamino-propan-2-ol, bis-(2-hydroxy-5-aminophenyl)methane, 1,3-bis-(2,5-diamino-phenoxy)propan-2-ol, N,N′-bis-(4-aminophenyl)-1,4-diazacycloheptane, 1,10-bis-(2,5-diaminophenyl)-1,4,7,10-tetraoxadecane, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo-[1,2-a]-pyrazol-1-one and physiologically tolerated salts and mixtures thereof. Preferred physiologically acceptable salts of the oxidation dye precursors comprising one or more amine groups are the hydrochlorides (monohydrochloride×HCl, or dihydro-chloride×2 HCl), the sulfate (×H₂SO₄), and the hydrobromides (monohydrobromide×HBr, or dihydro-bromide×2 HBr) of the compound.

Coupler-type oxidation dye precursors preferred as contemplated herein are selected from the group formed from 5-amino-2-methylphenol, 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-di-aminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-yl-phenyl)amino]ethanol, 3-amino-4-(2-methoxyethoxy)-5-methylphenyl amine, 1-amino-3-bis-(2-hydroxy-ethyl)aminobenzene, resorcinol, 2-methyl resorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or mixtures of these compounds or their physiologically tolerated salts.

In a preferred embodiment, a cosmetic dye composition (DC) comprises, as oxidation dye precursor, at least one developer component selected from the group of p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, 2-methoxymethyl-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diamino-propan-2-ol, bis-(2-hydroxy-5-aminophenyl)methane, 1,3-bis-(2,5-diaminophenoxy)-propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxy-ethyl)phenol and 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine or the physiologically tolerated salts of these compounds, and additionally at least one coupler component selected from the group of 3-aminophenol, 5-amino-2-methylphenol 3-amino-2-chloro-6-methylphenol, 2-hydroxy-4-aminophenoxyethanol, 5-amino-4-chloro-2-methylphenol, 5-(2-hydroxyethyl)amino-2-methylphenol, 2,4-dichloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diaminophenyl)propane, 2,6-bis(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or the physiologically tolerated salts of the abovementioned compounds. To provide a sustainable and environmentally friendly product, as little packaging material as possible should be used. For this reason, the agents as contemplated herein are preferably concentrates. Accordingly, the cosmetic product preferably comprises the oxidation dye precursor(s) in high total amounts, which, based on the total weight of the product, range from about 1.0 to about 60.0% by weight, preferably from about 1.5 to about 50.0% by weight, more preferably from about 1.7 to about 40.0% by weight, and most preferably from about 2.0 to about 35.0% by weight.

In a particularly preferred embodiment, a cosmetic product comprises—based on its total weight—one or more oxidation dye precursors in a total amount of from about 1.0 to about 60.0% by weight, preferably from about 1.5 to about 50.0% by weight, more preferably from about 1.7 to about 40.0% by weight and most preferably from about 2.0 to about 35.0% by weight. The oxidation dye precursors are part of the cosmetic dyeing composition (DC).

In cosmetic products preferred as contemplated herein and dyeing processes preferred as contemplated herein, the dyeing composition (DC) comprises one or more direct dyes. Preferred nonionic direct dyes are selected from the group of HC Yellow 2, HC Yellow 4, HC Yellow 5, HC Yellow 6, HC Yellow 12, HC Orange 1, Disperse Orange 3, HC Red 1, HC Red 3, HC Red 7, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, 1,4-diamino-2-nitrobenzene, 2-amino-4-nitrophenol, 1,4-bis(2-hydroxyethyl)amino-2-nitrobenzene, 3-nitro-4-(2-hydroxyethyl)aminophenol, 2-(2-hydroxyethyl)amino-4,6-dinitro-phenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 4-[(3-hydroxypropyl)amino]-3-nitrophenol, 4-nitro-o-phenylenediamine, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Preferred anionic direct dyes are selected from the group of Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, Bromophenol Blue and Tetrabromophenol Blue.

Suitable cationic direct dyes are cationic triphenylmethane dyes, such as Basic Blue 7, Basic Blue 26, Basic Violet 2 and Basic Violet 14, aromatic systems substituted with a quaternary nitrogen group, such as Basic Yellow 57, Basic Red 76, Basic Blue 99, Basic Brown 16 and Basic Brown 17, cationic anthraquinone dyes such as HC Blue 16 (Bluequat B) and direct dyes comprising a heterocycle comprising at least one quaternary nitrogen atom, in particular Basic Yellow 87, Basic Orange 31 and Basic Red 51. The cationic direct dyes marketed under the trademark Arianor are also suitable cationic direct dyes as contemplated herein.

The cosmetic product as contemplated herein may comprise—based on its total weight—one or more alkalizing agents selected from the group of (earth) alkali metal silicates, (earth) alkali metal metasilicates, (earth) alkali metal hydroxides, (earth) alkali metal phosphates and (earth) alkali metal hydrogen phosphates and basic amino acids in a total amount of from about 5.0 to about 60.0 wt. %, preferably from about 10.0 to about 55.0% by weight, further preferably from about 15.0 to about 50.0% by weight and most preferably from about 20.0 to about 45.0% by weight.

Preferably, the alkalizing agent(s) are selected from sodium silicates and sodium metasilicates, each having a molar SiO₂/Na₂O ratio of from about 0.8 to about 3.7, preferably from about 1.0 to about 3.6, more preferably from about 2.5 to about 3.5.

In further cosmetic products preferred as contemplated herein and dyeing processes preferred as contemplated herein, the dyeing composition (DC) comprises basic magnesium carbonate (magnesium carbonate hydroxide) as secondary alkalizing agent. Basic magnesium carbonate (magnesium carbonate hydroxide) has several advantages in the context of the present disclosure: it has a weaker alkaline effect than the sodium silicates and sodium metasilicates, each of which has a molar SiO₂/Na₂O ratio of about 0.8 to about 3.7, preferably of about 1.0 to about 3.6, particularly preferably of about 2.5 to about 3.5, and thus buffers the alkalinity of the colorant ready for use. Adjusting the preferred pH of the ready-to-use colorant as contemplated herein with one of the above-mentioned sodium (meta)silicates alone can lead to undesirable foaming when preparing the application mixture. This is slowed down by the addition of basic magnesium carbonate (magnesium carbonate hydroxide). In addition, basic magnesium carbonate (magnesium carbonate hydroxide) has particularly good adsorption properties. This can be used to remove dust from the anhydrous dyeing composition (DC) with an oil. The adsorption of an oil added for dust removal binds the fine powder particles of the individual components of the dyeing composition (DC), so that a low-dust or dust-free powder is obtained. In addition, the ready-to-use colorant forms a fine creamy mass in the presence of basic magnesium carbonate (magnesium carbonate hydroxide).

In cosmetic products preferred as contemplated herein and coloring processes preferred as contemplated herein, the cosmetic coloring composition (DC) comprises about 5 to about 16% by weight, preferably about 6 to about 14% by weight, particularly preferably about 8 to about 11% by weight of basic magnesium carbonate (magnesium carbonate hydroxide), in each case based on the weight of the anhydrous coloring composition (DC).

A further preferred embodiment of the colouring process as contemplated herein is wherein the colouring agent ready for use comprises about 0.5 to about 2.0 wt. %, preferably about 0.6 to about 1.2 wt. %, particularly preferably about 0.7 to about 1.0 wt. % basic magnesium carbonate (magnesium carbonate hydroxide), in each case based on the weight of the colouring agent ready for use. In a further preferred embodiment of the cosmetic product as contemplated herein and of the coloring process as contemplated herein, the product comprises at least one excipient selected from fillers, anti-caking agents and drying agents, as well as mixtures of these excipients. These excipients are intended to prevent the powder components of the cosmetic product from clumping or caking.

Particularly preferred fillers are selected from sodium sulphate and sodium chloride and mixtures thereof. Sodium chloride is particularly preferred because it can enhance the oxidizing effect of the solid oxidant, especially sodium perborate or sodium percarbonate. This is particularly desirable for achieving light color tones. Particularly preferred anti-caking agents are selected from fumed silicas, precipitated silicas, diatomaceous earth, calcium phosphate, calcium silicates, aluminum oxide, magnesium oxide, magnesium carbonate, zinc oxide, stearates, and mixtures thereof. Particularly preferred desiccants are selected from sodium sulphate, sodium carbonate, magnesium sulphate, calcium chloride, precipitated silicas and fumed silicas and mixtures thereof. The use of sodium sulfate and silicon dioxide has proved to be particularly preferable.

Silicon dioxide can be used, for example, in the form of amorphous silica. This substance has the CAS numbers 7631-86-9 and 112926-00-8 and is commercially available under the trade name Sipernat 22 “Amorphous silica” from Evonik.

In another particularly preferred embodiment, an agent as contemplated herein comprises at least one filler selected from the group of sodium sulfate and silicon dioxide.

In another particularly preferred embodiment, an agent as contemplated herein comprises at least one filler selected from the group of sodium sulfate and silicon dioxide.

In another particularly preferred embodiment, an agent as contemplated comprises the fillers sodium sulfate and silicon dioxide. The fillers are preferably used in certain quantity ranges in the medium.

In another particularly preferred embodiment, a product as contemplated herein comprises—based on its total weight—about 10.0 to about 65.0% by weight, preferably about 15.0 to about 65.0% by weight, further preferably about 20.0 to about 60.0% by weight and very particularly preferably about 25.0 to about 55.0% by weight of sodium sulfate.

In a further particularly preferred embodiment, a product as contemplated herein comprises—based on its total weight—about 2.0 to about 30.0% by weight, preferably about 2.0 to about 26.0% by weight, further preferably about 2.0 to about 20.0% by weight and very particularly preferably about 2.0 to about 12.0% by weight of silicon dioxide.

In a preferred embodiment, the cosmetic product further comprises at least one, two or more thickening agents. If the oxidation composition and the coloring composition are present in separate receiving chambers of the envelope or in separate envelopes, the thickening agent may be only a component of the oxidation composition or only a component of the coloring composition. It is also possible that both oxidation composition and colouring composition have one, two or more thickening agents, in which case they may be the same or different.

When mixing the coating of the present disclosure in water, the mixture remains low viscosity in the first seconds of mixing, so that mixing can be performed easily and quickly. The viscosity then increases, and the ready-to-use dye can be easily applied to the keratinous fibers due to the higher viscosity. After the usual time required for mixing and application, a viscosity maximum is reached. Then already applied mixture does not drip from the hair. Since the combination of thickener and alkalizing agent preferably used as contemplated herein causes the viscosity of the ready-to-use agent to develop only gradually, the ready-to-use colorant that can be prepared from the product as contemplated herein is easier to handle.

To achieve this effect, the thickener is preferably selected from polysaccharides, which are preferably selected from anionic cellulose and microbial gum and mixtures thereof.

For the purposes of the present disclosure, the term microbial gum means a substance produced by microorganisms during sugar fermentation. Examples of microbial gums or microbial gums include scleroglucan gums, gellane gums, pullulan gums, curdlan gums, xanthans, grifolan gums, lentinane gums, schizophyllan gums, spirulinan gums and crestin gums.

Particularly well-suited microbial gums can be selected from the group of xanthan gum, scleroglucan gum, gellan gum, pullulan gum, curdlan gum, grifolan gum, lentinan gum, schizophyllan gum, and crestin gum, and xanthan gum is particularly preferred.

As contemplated herein, the term “xanthans” or “xanthan gum” refers to naturally occurring polysaccharides that can be obtained from sugar-containing substrates with the aid of bacteria of the genus Xanthomonas. Preferably, the xanthan gum used as contemplated herein comprises d-glucose, d-mannose, d-glucuronic acid, acetate and pyruvate in a molar ratio of about 28:30:20:17:5,1-6,3, with the main chain comprising β-1,4-linked glucose units (also known as the cellulose chain). The xanthanes particularly preferred in the present disclosure have CAS No. 11138-66-2 and the following structural formula:

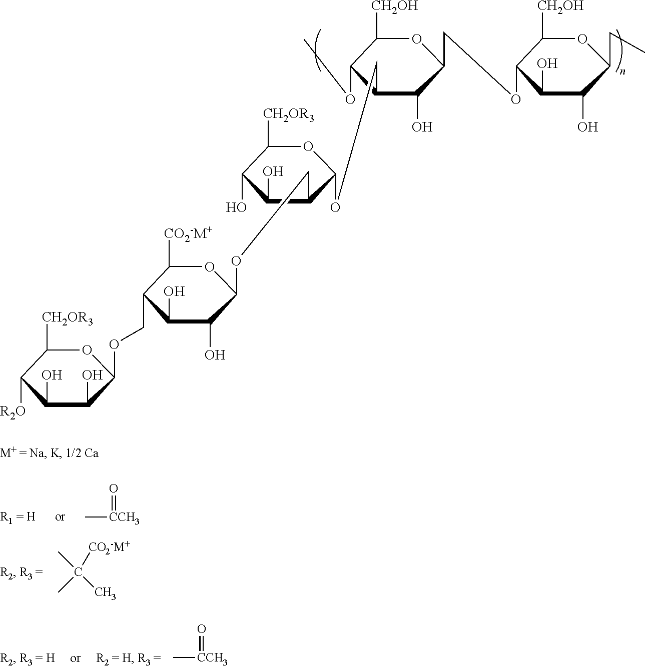

Xanthan gum is a polyelectrolyte due to the presence of carboxylate groups. In the context of the present disclosure, the use of xanthans, at least about 95 wt. % of which have a particle diameter of less than about 0.180 mm mesh size in powder form and whose about 1 wt. % solution in an aqueous about 1 wt. % KCl solution (potassium chloride) has a viscosity of about 1400 to about 1600 mPa s (measured with Brookfield LVTD, spindle 3, 60 rpm, 25° C.), has been shown to be particularly advantageous. Such xanthans are commercially available, for example, under the trade name Keltrol CG-SFT from CP Kelco. Gellan gum, which can also be referred to simply as gellan, is an unbranched anionic microbial heteroexopolysaccharide with a basic tetrasaccharidic unit comprising the monomer's glucose, glucuronic acid and rhamnose. Every basic unit is esterified with an 1-glycerate, and every other basic unit is esterified with an acetate. Gellan carries the CAS number 71010-52-1.

Pullulan gum, or pullulan for short, is a natural, water-soluble linear polysaccharide comprising maltotriose units. Three glucose units of maltotriose are linked by α-1,4-glycosidic linkages, whereas successive maltotriose units are linked by α-1,6-linkages. Pullulan is produced from starch and sugar with the help of the fungus Aureobasidium pullulans. Pullulan has the CAS number 9057-02-7.

Curdlan gum, or curdlan for short, is a homoglycan of glucose with glycosidic linkages of the β-(1,3) type formed by Agrobacterium biobar, Euglena gracilis, and Alcaligenes faecalis var. myxogenes, among others. In some species, paramylons with additional β-(1,6)-bonds occur in the periplasm. Curdlan carries the CAS number 51052-65-4.

Grifolan gum or grifolan for short is a (1→3;1→6)-β-d-glucan from Grifola frondosa (Basidiomycota, Agariomycetes). Grifolan is a (1→3;1→6)-β-d-glucan related to scleroglucan.

Lentinan gum, or lentinan for short, is produced by the microbes from Lentinus edodes (sawfly, shiitake). Lentinan is a (1→3;1→6)-β-d-glucan related to scleroglucan. Lentinan is a substance isolated from the shiitake mushroom (Lentinula edodes). Chemically, it is a glucan, with two β-1,6-glycosidic branches for every five straight-chain β-1,3-glycosidically linked monomers. Lentinan carries the CAS number 37339-90-5.

Schizophyllan gum or schizophyllan for short is a (1→3;1→6)-β-d-glucan from Schizophyllum commune (Basidiomycota, Agariomycetes). Schizophyllan is a (1→3;1→6)-β-d-glucan related to scleroglucan.

Krestin gum or Kresti for short is a 3-glucan protein complex (proteoglycan) from the white rot fungus Coriolus versicolor CM-101 (Syn. Trametes versicolor, Polyporus versicolor; Basidiomycota). It is obtained by extracting the mycelium of submerged cultures with hot water and precipitating with saturated ammonium sulphate solution.

To produce the optimum thickening effects, the microbial gum(s) are preferably used in specific ranges of amounts in the product of the present disclosure. Fast, reproducible and uniform thickening, even at high salt contents, could be achieved if the cosmetic product included in the coating as contemplated herein comprises—based on its total weight—one or more microbial gums in a total amount of about 1.5 to about 12.0% by weight, preferably of about 2.0 to about 10.0% by weight, more preferably of about 3.0 to about 8.0% by weight and very preferably of about 4.5 to about 6.5% by weight.

Although some basic thickening can be achieved with the use of at least one microbial gum, it has been found that the microbial gum is often insufficient as the sole thickener in the products of the present disclosure. It has been shown that optimum, rapid thickening can be achieved if the compositions as contemplated herein comprise, in addition to microbial gum, at least one further thickener which is an anionic cellulose.

Cellulose is the main component of plant cell walls (about 50% by mass) and thus the most common organic compound and the most common polysaccharide. It is unbranched and comprises several hundreds to tens of thousands of (β-1,4-glycosidically linked) β-D-Glucose- or Cellobiose-Units. Cellulose is a Polymer (polysaccharide ‘multiple sugar’) from the monomer cellobiose, which in turn is a Disaccharide (‘twofold sugar’) and Dimer of the Monosaccharide (‘simple sugar’) glucose. The monomers are linked by β-1,4-glycosidic bonds. In the solid state, crystalline regions in cellulose alternate with those of low order (amorphous regions). Natural and manufacturing-related impurities, such as the presence of carboxy groups, are typically in the range of approx. 1%. As contemplated herein, cellulose itself is therefore not considered an anionic polysaccharide.

An anionic cellulose is understood to be a derivative of cellulose that carries at least one negative charge. This negative charge can come about, for example, through a deprotonated carboxyl group —COO⁻, a deprotonated sulfonyl group —SO₃⁻ or a deprotonated phosphonate group —P(O)O₂²—. To maintain charge neutrality, these anionic groups are balanced by the presence of the appropriate amounts of cationic counterions such as Na⁺ ions, K⁺ ions or ammonium ion NH4)⁺. Examples of carboxylate-containing monomers are carboxyalkyl ethers of sugars and sugar acids (uronic acids).

Anionic celluloses preferred as contemplated herein are carboxy-C₁-C₆-alkyl celluloses, which are preferably used in the form of their physiologically compatible salts. Physiologically compatible salts are the ammonium salts as well as the salts of alkali metals, in particular sodium and potassium, alkaline earth metals, in particular magnesium and calcium, as well as of zinc.

Carboxy-C₁-C₆-alkyl celluloses are cellulose ethers, i.e., derivatives of cellulose in which part of the hydroxyl groups are linked as ethers to a carboxy-C₁-C₆-alkyl group (—CH₂)ₙ—COOH). The index number n here indicates the number of —CH₂ groups, which can range from about 1 to about 6.

As a very particularly preferred anionic polysaccharide, carboxymethylcellulose is used either in free form or in the form of its physiologically compatible salt, especially its sodium salt. In its acid form, carboxymethyl cellulose carries the CAS number 9000-11-7. The sodium salt of carboxymethyl cellulose has the CAS number 9004-32-4.

In cosmetic products preferred as contemplated herein and dyeing processes preferred as contemplated, the dyeing composition (DC) comprises, in each case based on its weight, about 5 to about 25% by weight, preferably about 10 to about 22% by weight, particularly preferably about 12 to about 19% by weight. %, extremely preferably about 13 to about 18% by weight of anionic celluloses, in particular sodium carboxymethyl cellulose.

In further cosmetic products preferred as contemplated herein and coloring processes preferred as contemplated herein, the coloring composition (DC) comprises, in each case based on its weight, about 2 to about 10% by weight, preferably about 3 to about 8% by weight, particularly preferably about 4 to about 7% by weight, exceptionally preferably about 5.5 to about 6% by weight of xanthan gum.

In further cosmetic products preferred as contemplated herein and dyeing processes preferred as contemplated herein, the dyeing composition (DC) comprises, in each case based on its weight, about 5 to about 25% by weight, preferably about 10 to about 22% by weight, particularly preferably about 12 to about 19% by weight. %, exceptionally preferably about 13 to about 18% by weight of anionic celluloses, in particular sodium carboxymethyl cellulose, and about 2 to about 10% by weight, preferably about 3 to about 8% by weight, particularly preferably about 4 to about 7% by weight, exceptionally preferably about 5.5 to about 6% by weight of xanthan gum.

In further cosmetic products preferred as contemplated herein and dyeing processes preferred as contemplated herein, the dyeing composition (DC) comprises as thickening agent a mixture of anionic celluloses, in particular sodium carboxymethyl cellulose, and microbial gum, in particular xanthan gum, the weight ratio of anionic celluloses to microbial gum being in the range of from about 10.0 to about 1.0, preferably from about 9.0 to about 1.5, more preferably from about 8.0 to about 2.0, still more preferably from about 6.0 to about 2.5 and most preferably from about 5.0 to about 3.0. In other words, it has been found to be particularly advantageous if the anionic celluloses, especially the sodium carboxymethyl cellulose, were used in an excess of about 1 to about 10 times the weight of the microbial gum, especially the xanthan gum. The best results were obtained when the anionic cellulose was incorporated into the compound in a three-to fivefold excess by weight compared to the microbial gum.

In a further preferred embodiment of the dyeing process as contemplated herein, the ready to use colorant comprises about 0.4 to about 3.0% by weight, preferably about 0.8 to about 2.6% by weight, particularly preferably about 1.2 to about 2.2% by weight of anionic celluloses, in particular sodium carboxymethyl cellulose, in each case based on the weight of the ready to use colorant.

In a further preferred embodiment of the dyeing process as contemplated herein, the ready to use colorant comprises about 0.1 to about 1.2% by weight, preferably about 0.3 to about 1.0% by weight, particularly preferably about 0.4 to about 0.6% by weight of xanthan gum, in each case based on the weight of the ready to use colorant.

In a further preferred embodiment of the dyeing process as contemplated herein, the ready to use dyeing composition comprises about 0.4 to about 3.0% by weight, preferably about 0.8 to about 2.6% by weight, particularly preferably about 1.2 to about 2.2% by weight of % by weight of anionic celluloses, in particular sodium carboxymethyl cellulose, and about 0.1 to about 1.2% by weight, preferably about 0.3 to about 1.0% by weight, particularly preferably about 0.4 to about 0.6% by weight of xanthan gum, in each case based on the weight of the colorant ready for use.

In a preferred embodiment, the cosmetic product for the oxidative colouring of keratinous fibers, in particular human hair, thus comprises
(i) at least one oxidizing composition comprising at least one perborate or at least one percarbonate as solid oxidizing agent,
(ii) at least one cosmetic dye composition comprising at least one developer-type oxidation dye precursor and at least one coupler-type oxidation dye precursor,
(iii) at least one microbial gum, in particular xanthan gum,
(iv) at least one anionic cellulose, in particular sodium carboxymethyl cellulose, and
(v) less than about 10% by weight of water,
where the percentage by weight is based on the total weight of the product.
   In summary, very particularly preferred is a cosmetic product for the oxidative coloring of keratinous fibers, in particular human hair, comprising, based on its total weight
(i) about 0.5 to about 14.0% by weight sodium perborate,
(ii) one or more oxidation dye precursors in a total amount of 1.0 to 60.0% by weight
(iii) about 1.5 to about 12.0% by weight xanthan gum,
(iv) about 10.0 to about 28.0% by weight of sodium carboxymethyl cellulose, and
(v) less than about 10 wt. %, preferably about 0.5 to about 3.0 wt. % water.

In further cosmetic products preferred as contemplated herein and coloring processes preferred as contemplated herein, no ammonium salt is present. In other cosmetic products preferred as contemplated herein and dyeing processes preferred as contemplated herein, no polymer or copolymer with acrylate-, methacrylate- or vinyl-containing monomers and no polyurethane is present.

Anhydrous oxidation compositions (OC) preferred as contemplated herein and anhydrous dyeing compositions (DC) preferred as contemplated herein each have a bulk density in the range of about 450 to about 1000 g/l (gram/liter), preferably about 500 to about 950 g/l, particularly preferably about 550 to about 900 g/l. The bulk density is preferably determined according to EN ISO 600 DIN 53468. Unless otherwise stated, all temperature data refer to a pressure of 1013 mbar.

A further preferred embodiment of the dyeing process as contemplated herein is wherein the ready-to-use dye has a viscosity in the range of about 10 to about 100 Pa s, preferably about 20 to about 85 Pa s, particularly preferably about 40 to about 80 Pa s, exceptionally preferably about 60 to about 75 Pa s, in each case measured at 20° C. with a Brookfield rotational viscometer, model LVDVII+, spindle 5, 4 revolutions per minute (rpm).

Further cosmetic products preferred as contemplated herein and dyeing processes preferred as contemplated herein are wherein the anhydrous oxidation composition (OC) and/or the anhydrous dyeing composition (DC) additionally comprise a dedusting agent which prevents dust formation of the powdery ingredients. Oils can be used as dedusting agents, especially oils selected from kerosene oil, silicone oil or ester oil as well as mixtures of these oils, kerosene oil being particularly preferred.

Further cosmetic products preferred as contemplated herein and coloring processes preferred as contemplated herein are wherein the coloring composition (DC) comprises, in each case based on its weight, at least one oil in a total amount of about 0.1 to about 10.0% by weight, preferably about 1.0 to about 6.0% by weight, particularly preferably about 2.0 to about 3.0% by weight.

Further cosmetic products preferred as contemplated herein and coloring processes preferred as contemplated herein are wherein the coloring composition (DC) comprises, in each case based on its weight, kerosene oil in a total amount of about 0.1 to about 10.0% by weight, preferably about 1.0 to about 6.0% by weight, particularly preferably about 2.0 to about 3.0% by weight.

Further cosmetic products preferred as contemplated herein and dyeing processes preferred as contemplated herein are wherein the anhydrous dyeing composition (DC) comprises one or more acids which are present as a solid under standard conditions. Such an acid, in combination with the alkalizing agent, allows improved buffering of the pH of the ready-to-use colorant. It is therefore preferred in the context of the present disclosure that the anhydrous dyeing composition (DC) additionally comprises at least one acid selected from the group of succinic acid, dipicolinic acid, citric acid, malic acid, maleic acid and/or tartaric acid.

Oxidative staining processes on keratin fibers usually take place in an alkaline environment. To protect the keratin fibers and the skin as much as possible, the adjustment of a too high pH-value is however not desirable. It is therefore preferred if the pH of the ready-to-use colorant which can be prepared from the cosmetic product as contemplated herein or preferred as contemplated herein is in the range from about 7 to about 11, preferably in the range from about 8 to about 11.0, particularly preferably in the range from about 9 to about 10.5, exceptionally preferably in the range from about 9.3 to about 10.0, in each case measured at a temperature of about 20° C.

The anhydrous dyeing composition (DC) may further comprise additional active ingredients, auxiliaries and additives. For example, it may comprise one or more fat components from the group of C₁₂-C₃₀-Fatty alcohols C₁₂-C₃₀-Fatty acid triglycerides, C₁₂-C₃₀-Fatty acid monoglycerides, C₁₂-C₃₀-Fatty acid diglycerides and/or hydrocarbons.

Preferably, the anhydrous dyeing composition (DC) may additionally comprise a surface-active substance, such surface-active substances being referred to as surfactants or as emulsifiers, depending on the field of application. They are preferably selected from anionic, zwitterionic, amphoteric and non-ionic surfactants and emulsifiers.

Furthermore, the anhydrous dyeing composition (DC) may comprise other active ingredients, auxiliaries and additives, such as non-ionic polymers, for example polyethylene glycols solid at standard conditions, quaternized cellulose ethers, hair-conditioning compounds such as phospholipids, for example lecithin; Perfume oils, fiber structure improving agents, especially mono-, di- and oligosaccharides such as glucose, galactose, fructose, fructose and lactose; dyes for coloring the agent; amino acids and oligopeptides; protein hydrolysates on animal and/or vegetable basis, as well as in the form of their fatty acid condensation products or optionally anionically or cationically modified derivatives; fatty substances and vegetable oils; active ingredients such as panthenol, pantothenic acid, pantolactone, pyrrolidinonecarboxylic acids and their salts; ceramides or pseudoceramides; vitamins, provitamins and vitamin precursors; plant extracts; fats and waxes such as fatty alcohols, beeswax, montan wax and kerosenes.

### EXAMPLES

The present disclosure is explained in more detail based on the following examples, which are by no means to be understood as a limitation of the inventive idea.

The following bags of non-woven polyvinyl alcohol were provided:
i) cross-laid and spot-welded non-woven fabric with a basis weight of 28 g/m²;
ii) cross-laid and spot-welded non-woven fabric with a basis weight of 28 g/m²;
iii) woven nonwoven fabric with a basis weight of 60 g/m²;
(iv) woven non-woven fabric with a basis weight of 100 g/m²;
(v) woven non-woven fabric with a basis weight of 140 g/m²;
   and each filled with a cosmetic product of the composition summarized in Table 1 in powder form. Suitable nonwovens are available, for example, under the trade names DO 102 and DO 105 from Freudenberg Performance Materials Holding SE & Co. KG, Germany.

TABLE 1
(all quantities in grams):

| | Example 1 | Example 2 | Example 3 |
|---|---|---|---|
| Cosmetic preparation | 100.00 | 100.00 | 100.00 |
| Sodium perborate | 2.00 | 4.00 | 6.00 |
| Sodium carbonate | 2.00 | 2.00 | 2.00 |
| basic magnesium carbonate | 8.50 | 8.50 | 8.50 |
| (magnesium carbonate hydroxide) | | | |
| heavy pharm. 400 g/l | | | |
| Sipernate 22 (INCI: Silica, | 8.00 | 8.00 | 8.00 |
| precipitated silica) | | | |
| Aeroperl 300/30 (INCI: Silica, | 0.40 | 0.40 | 0.40 |
| fumed silica) | | | |
| p-Toluylenediamine sulfate | 19.70 | 19.70 | 19.70 |
| 2,4-Diaminophenoxyethanol 2 HCl | 1.60 | 1.60 | 1.60 |
| Resorcinol | 2.60 | 2.60 | 2.60 |
| m-Aminophenol | 6.60 | 6.60 | 6.60 |
| Sodium sulphate | 17.10 | 14.35 | 11.60 |
| Sodium metasilicate anhydrous* | 6.00 | 6.00 | 6.00 |
| Sodium carboxymethyl cellulose | 18.00 | 18.50 | 19.00 |
| (INCI: Cellulose gum; Cekol 50000) | | | |
| Xanthan | 5.00 | 5.25 | 5.50 |
| Paraffinum Liquidum | 2.50 | 2.50 | 2.50 |
| PVA non-woven (packaging material) | 0.5 | 0.5 | 0.5 |

*Sodium metasilicate anhydrous: Silmaco powder FE, molar SiO₂/Na₂O ratio: 0.91-1.01 The bags comprising the preparation according to Table 1 were dissolved in water. The weight ratio of the product to water was 2.2. The bag as well as the powder dissolved without leaving any residue.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A portion unit comprising at least one receiving chamber which is formed by a water-soluble envelope, wherein the water-soluble envelope comprises a water-soluble nonwoven fabric and wherein the receiving chamber comprises a cosmetic product for the oxidative color change of keratinous fibers, the cosmetic product comprising (i) at least one oxidation composition (OC) comprising at least one solid oxidizing agent as oxidizing compound, and (ii) at least one cosmetic coloring composition (DC).

2. A portion unit according to claim 1, wherein the water-soluble nonwoven is selected from the group of needle punched nonwovens.

3. A portion unit according to claim 1, wherein the water-soluble nonwoven fabric is a non-woven, cross-laid and spot-sealed nonwoven fabric.

4. A portion unit according to claim 1, wherein the water-soluble nonwoven comprises at least about 50% by weight of polyvinyl alcohol or polyvinyl alcohol polymers based on the total weight of the water-soluble coating.

5. A portion unit according to claim 1, wherein the solid oxidant of the anhydrous oxidation composition (OC) is selected from the group of percarbonates, perborates, percarbamides or mixtures thereof.

6. A portion unit according to claim 1, wherein the cosmetic product further comprises at least one thickening agent.

7. A portion unit according to claim 1, wherein the cosmetic product further comprises at least one alkalizing agent selected from the group of sodium silicates and sodium metasilicates, each having a molar SiO₂/Na₂O ratio of from about 0.8 to about 3.7.

8. A portion unit according to claim 1, wherein the dyeing composition (DC) comprises at least one developer-type oxidation dye precursor (developer component) and at least one coupler-type oxidation dye precursor (coupler component), wherein the at least one developer component is present in a total amount of about 0.1 to about 40% by weight.-%, based on the weight of the anhydrous dyeing composition (DC), and the at least one coupler component is present in an amount of from about 0.1 to about 40% by weight.-% based on the weight of the anhydrous dyeing composition (DC).

9. A portion unit according claim 1, wherein the dyeing composition (DC) comprises one or more direct dyes.

10. A cosmetic process for oxidative color change of keratinous fibers comprising the following steps:
(i) providing a portion unit of claim 1,
(ii) mixing the portion unit with water to a ready-to-use colorant,
(iii) directly following step (ii), applying the ready-to-use colorant to keratinous fibers,
(iv) leaving the ready-to-use colorant on the keratinous fibers for about 5 to about 60 minutes, and
(v) rinsing the ready-to-use colorant from the keratinous fibers with water,
wherein the weight ratio of the weight of the cosmetic product to the weight of the amount of water used in step (ii) is from about 16:100 to about 25:100.

11. A portion unit according to claim 2, wherein the water-soluble nonwoven fabric is a non-woven, cross-laid and spot-sealed nonwoven fabric.

12. A portion unit according to claim 2, wherein the water-soluble nonwoven comprises at least 50% by weight of polyvinyl alcohol or polyvinyl alcohol polymers based on the total weight of the water-soluble coating.

13. A portion unit according to claim 3, wherein the water-soluble nonwoven comprises at least 50% by weight of polyvinyl alcohol or polyvinyl alcohol polymers based on the total weight of the water-soluble coating.

14. A portion unit according to claim 2, wherein the solid oxidant of the anhydrous oxidation composition (OC) is selected from the group of percarbonates, perborates, percarbamides or mixtures thereof.

15. A portion unit according to claim 3, wherein the solid oxidant of the anhydrous oxidation composition (OC) is selected from the group of percarbonates, perborates, percarbamides or mixtures thereof.

16. A portion unit according to claim 4, wherein the solid oxidant of the anhydrous oxidation composition (OC) is selected from the group of percarbonates, perborates, percarbamides or mixtures thereof.

17. A portion unit according to claim 1, wherein
the water-soluble envelope consists of the water-soluble nonwoven fabric;
the receiving chamber comprises the cosmetic product for the oxidative color change of human hair;
the at least one oxidation composition (OC) is anhydrous;
the water-soluble nonwoven is selected from the group of needle punched nonwovens;
the water-soluble nonwoven fabric is a non-woven, cross-laid and spot-sealed nonwoven fabric that is single-layered;
the water-soluble nonwoven comprises at least 98% by weight of polyvinyl alcohol based on the total weight of the water-soluble coating;
the solid oxidant of the anhydrous oxidation composition (OC) is selected from sodium percarbonate, sodium perborate, and combinations thereof;
the cosmetic product further comprises a polysaccharide selected from sodium carboxymethyl cellulose and xanthans and mixtures thereof;
the cosmetic product further comprises at least one alkalizing agent selected from sodium silicates, sodium metasilicates, and combinations thereof, having a molar SiO₂/Na₂O ratio of from about 2.5 to about 3.5;
the dyeing composition (DC) comprises at least one developer-type oxidation dye precursor (developer component) and at least one coupler-type oxidation dye precursor (coupler component),
the at least one developer component is selected from toluene-2,5-diamine, 2-methoxymethyl-p-phenylenediamine, 2-(2,5-diaminophenyl)ethanol, p-phenylenediamine, 2-(1,2-dihydroxyethyl)-p-phenylenediamine, N,N-bis-(2-hydroxyethyl)-p-phenylenediamine, 4,5-diamino-1-(2-hydroxyethyl)-1H-pyrazole, 4-aminophenol, 3-methyl-4-aminophenol, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diamino-propan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, 2,4,5,6-tetraaminopyrimidine, 4-hydroxy-2,5,6-triaminopyrimidine, 2-hydroxy-4,5,6-triaminopyrimidine, 2,3-diamino-6,7-dihydro-1H,5H-pyrazolo-[1,2-a]-pyrazol-1-one and physiologically tolerated salts and mixtures thereof, in a total amount of about 10 to about 20 wt. % based on the weight of the anhydrous dyeing composition (DC), and
the at least one coupler component is selected from 5-amino-2-methylphenol, 3-amino-2-chloro-6-methylphenol, 2-hydroxy-4-amino-phenoxyethanol, 5-amino-4-chloro-2-methylphenol, 5-(2-hydroxyethyl)-amino-2-methylphenol, 2,4-di-chloro-3-aminophenol, 2-aminophenol, 3-phenylenediamine, 2-(2,4-diaminophenoxy)ethanol, 1,3-bis(2,4-diaminophenoxy)propane, 1-methoxy-2-amino-4-(2-hydroxyethylamino)benzene, 1,3-bis(2,4-diamino-phenyl)propane, 2,6-bis(2′-hydroxyethylamino)-1-methylbenzene, 2-({3-[(2-hydroxyethyl)amino]-4-meth-oxy-5-methyl¬phe-nyl}amino)ethanol, 2-({3-[(2-hydroxyethyl)amino]-2-methoxy-5-methylphenyl}-amino)ethanol, 2-({3-[(2-hydroxyethyl)amino]-4,5-dimethylphenyl}amino)ethanol, 2-[3-morpholin-4-yl-phenyl)amino]ethanol, 3-amino-4-(2-methoxyethoxy)-5-methylphenylamine, 1-amino-3-bis-(2-hydroxyethyl)aminobenzene, resorcinol, 2-methylresorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthol, 1,5-di¬hydroxy¬naphtha-lin, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or mixtures of these compounds or their physiologically tolerated salts, in a total amount of about 6 to about 10% by weight, based on the weight of the anhydrous dyeing composition (DC); and
the dyeing composition (DC) comprises one or more direct dyes.
